(19) United States
(12) Patent Application Publication
(10) Pub. No.: US 2007/0065497 A1
(43) Pub. Date: Mar. 22, 2007

(54) Combination and method using EDTA combined with glutathione in the reduced state encapsulated in a liposome to facilitate the method of delivery of the combination as an oral, topical, intraoral or transmucosal, for anti-thrombin effect and for anti-platelet aggregation and measurement of efficacy

(76) Inventor: Frederick Timothy Guilford, Palo Alto, CA (US)

Correspondence Address: BROOKE SCHUMM III, ONE NORTH CHARLES STREET, SUITE 2450, BALTIMORE, MD, 21201, US

(21) Appl. No.: 11/230,277

(22) Filed: Sep. 20, 2005

(51) Int. Cl. A61K 38/05; A61K 9/127; A61K 31/195; A61K 33/04; A61K 33/32

(52) U.S. Cl. 424/450; 424/641; 424/702; 514/18; 514/566

(57) ABSTRACT

The invention is for the combination of EDTA and glutathione in the reduced state encapsulated in a liposome for prevention and therapy of coronary artery disease (CAD), and anti-thrombotic effect and for the effect of restoring platelet aggregation, an integral component of thrombus formation, to normal. The combination is particularly efficacious for vascular deficiency ailments including atherosclerotic vascular disease, reduction of ischemic cerebral event, complications from surgical procedures including re-stenosis, neurodegenerative disease, erectile dysfunction, and vascular deficiency resulting from etiology of sepsis and chronic infection and diabetes. Additionally the invention is used as an adjunct in the management of diabetes for both the slowing and amelioration of the atherosclerotic state and as an adjunct with standard diabetic medications such as insulin for the lowering of glucose levels.

### SUMMARY OF INVENTION

The invention proposes the use of ethylene diamine tetraacetic acid (“EDTA”) and glutathione in the reduced state encapsulated in a liposome for treatment of vascular deficiency diseases, or vascular insufficiency in other diseases such as sepsis or chronic infection. Heading

### CONTINUATION DATA

This is a continuation in part of provisional application 60/123456 filed on Sep. 16, 2004 with the title of Combination and method using EDTA combined with glutathione in the reduced state encapsulated in a liposome to facilitate the method of delivery of the combination as an oral, topical, intra-oral or transmucosal for anti-thrombin effect and for anti-platelet aggregation and measurement of efficacy.

### FIELD OF THE INVENTION

The present invention relates to the field of administration of a liposome containing a combination of reduced glutathione and EDTA to maintain antioxidant function and remove transition metal that contribute to the formation of disease states such as atherosclerosis, Parkinson's disease and diabetes.

### BACKGROUND

Vascular disease, in particular coronary artery disease (CAD) is the leading killer of men and women in the western world. The costs of CAD are staggering, reaching approximately $325 billion per year in 2002. Vascular problems, including those triggered by inflammatory processes, further contribute to an array of vertebrate afflictions. Interventions by vascular procedures, while potentially salutary in effect in the longer term, often stimulate inflammatory responses. Absent appropriate intervention with ethylene diamine tetraacetic acid (“EDTA”) in combination with glutathione encapsulated in a liposome to facilitate absorption into the system a number of deleterious events occur, including the inducement of the coagulation system to undergo a metamorphosis which leads to a “sticky” effect accompanied by activation of the intrinsic coagulation cascade.

EDTA ethylenediaminetetraacetic acid is a synthetic molecule known for complexing metal ions. EDTA is capable of forming a complex with +2 and higher metal ions in a 1:1 metal to EDTA complex. EDTA has been used in foods to stabilize metals for a variety of reasons such as binding oxidized iron to slow the oxidation or deterioration of food. EDTA has a very high affinity for lead and is approved by the Food and Drug Administration (FDA) as a treatment for lead poisoning. This has previously been done by infusing the EDTA in an intravenous infusion. The EDTA complex with the metal is excreted through the kidney resulting in a decrease in the metal in the body.

The tripeptide L-glutathione (GSH) (gamma-glutamyl-cysteinyl-glycine) is well known in biological and medical studies to serve several essential functions in the cells of higher organisms such as mammals. It is functional when it appears in the biochemical form known as the reduced state (GSH). When oxidized, it forms into a form known as a dimer (GSSG), and after interaction with a reducing agent such as vitamin C, can returned to the active state, without loss of function.

As our understanding of vascular disease increases the mechanisms that lead to artery blockage has been expanded to include several mechanisms including the observation that vascular insufficiency has been recently defined as an inflammatory disorder. In the 1990's vascular disease was viewed as being caused by the slowly progressive deposition of fat along arteries, or a straight forward plumbing problem. While the effect of narrowing in the arterial pipes is true and is the basis of vascular disease, a more complex picture of the cause of vascular disease is emerging. As we will see, the new picture of vascular disease is moving the concept away from a mechanical blockage to a metabolic disorder. At the same time, the focus is moving away from mechanical solutions to metabolic solutions for vascular disease.

Initially, vascular disease develops with the deposition of excess cholesterol and triglycerides onto the artery walls. Special immune cells in the area ingest the excess fats and migrate to the layer under the lining of the artery, creating a small “bump” in the artery. If the process of fat deposition and plaque production continues it can expand the inner wall of the artery into the lumen of the artery. If this process progresses long enough a blockage is formed that can reduce the blood flow through the artery, resulting in muscle due to decreased oxygenation and often presenting as chest pain known as angina pectoris.

Prior to the year 2000 the perspective on vascular disease postulated the risk from plaque build up was due to the fact that cholesterol related plaques simply built up to the point that they would create blockage and obstruct blood flow. The Framingham Heart Study showed that as many as one third of all coronary heart disease (CHD) events occurred in individuals with total cholesterol <200 mg/dL. Considering that the average U.S. cholesterol level is approximately 210 to 220 mg/dL, almost half of all heart attack events and all stroke events that will occur in the United States next year will in fact occur among individuals with below-average lipid levels. For this reason, research groups have sought in large-scale prospective epidemiologic studies to get a better understanding of other markers associated with cardiovascular risk (Castelli W P). http://www.lipidsonline.org/slides/slide01.cfm?q=inflammation&dpg=2

The Real Threat

Recently it has been determined that the real threat of sudden death from artery disease comes from plaques that are weakened by inflammation and rupture under the stress of blood flow and blood pressure. These unstable plaques build up without warning symptoms and can rupture without warning. The disruption of these plaques can cause a massive blood clot that can suddenly obstruct the artery (Johnson, ABC News). The elements that create this series of events are being increasingly understood and include the fact that the dangerous plaques have a thin covering and are filled with inflammation stimulating material that makes them likely to burst without warning.

Plaque develops from fat that is deposited on the lining surface of blood vessels. Arteries actually have several layers including an outer, tough containment layer, a muscular inner layer and a layer of smooth epithelial cells also known as endothelial cells. The trigger for fat deposition may happen in response to minute inflammatory irritations on the lining of the vessel. In an attempt to remove the fat, white blood cells called macrophages attach to the fatty accumulation, ingest or absorb it and migrate to the inner lining of arteries. The fat is now in the macrophages lying just below the lining of the artery. Under the right conditions, these cells can then break the fat down and return it to the liver via the high density lipoproteins known as HDL.

Much depends on the condition of the fat that is ingested by macrophages. If the fat, known as Low Density Lipoprotein, LDL, is oxidized (rancid fat is oxidized fat) and stays in this state, it activates or triggers a continuing inflammatory response (Aviram).

Atherosclerosis is a Systemic Disease

“When you have a heart attack, the area that emerges is the tip of the iceberg,” explains the lead author of a study on vulnerable plaque, James A. Goldstein, a cardiologist at the William Beaumont Hospital in Royal Oak, Mich. (Plaque Attacks). “There is corrosion in the pipes throughout the house.” Dr. Goldstein was quoted in the national news, September 2000, after reporting a study that demonstrates that 40% of individuals with a heart attack had several unstable plaques remaining in their coronary vessels (Goldstein). Patients with the multiple plaques are more likely to experience a repeat heart attack within the year. They are also more likely to develop symptoms that require repeated angioplasty or bypass surgery. Dr. Sergio Waxman, a cardiologist at the University of Texas Medical Branch in Galveston has pointed out that even if all the unstable plaque lesions could be found it may not be possible to stint or shunt all of them (Plaque Attack).

Risk of plaque disruption depends more on plaque vulnerability and thrombogenicity than on plaque size or the severity of stenosis (Banas). Understanding these mechanisms is important to understanding how to decrease their risk. As atherosclerosis is now being viewed as a systemic disease (Nappi), markers are being sought that identifies those individuals at greater risk (Johnson).

One established marker of inflammation is a protein released from the liver during inflammation in the body called C reactive protein, or CRP. It has been observed that increased coagulation accompanies elevations of CRP. One of the changes associated with elevated CRP and chronic inflammation is a generalized increase in blood coagulation and platelet aggregation. These findings are also being identified in the formation of vascular disease. There are suggestions that low grade infections may trigger some of the events involved in creating arterial plaque. The development of the inflammatory or infectious triggers and subsequent events are being increasingly identified.

The development of atherosclerosis is being attributed to an interplay among several factors, including endothelial cell damage, adherence and activation of platelets, increased levels of lipids, oxidation of lipoproteins, infiltration of the vessel wall with macrophages with their subsequent conversion to fat or lipid laden cells that look foamy under microscopic examination and are called foam cells. These changes are accompanied by smooth muscle cell proliferation and migration leading to further thickening of the lining of arteries and narrowing of the interior of arteries. Activated platelets play a role in the progression of atherosclerosis through diverse pathways, and methods that inhibit platelet activation slow the formation of atherosclerotic lesions in several experimental systems (Rother).

The sequence of events in the development of arteriosclerosis progress, damage or irritation in the lining of an artery leads to the accumulation of excess LDL (low density lipoprotein) inside the artery wall. This leads to a thinning of the tissue separating the lipid from the inside of the artery. As this happens an inflammatory reaction is signaled throughout the body.

The ratio of Oxidants to Antioxidants play a role as oxidized LDL increases the erosion of plaque, releasing the triggers of the coagulation response (Li). Oxidized LDL is absorbed by white blood cells called macrophages that become the foam cells. The ability of the macrophages to eliminate excess cholesterol depends on the availability of adequate antioxidant function (Aviram). The foam cell migrates through the lining of the artery creating the swelling in the artery lining called plaque (Andrew). Oxidized LDL (Ox-LDL)-induced macrophage cholesterol accumulation and foam cell formation, the hallmark of early atherosclerosis (Kaplan). The atherosclerotic lesion consists of macrophages filled with cholesterol derived from oxidized-low density lipoprotein (Ox-LDL) and also contains platelet aggregates. The aggregation of platelets plays a key role in the formation of the atherosclerotic lesion and can be increased by the presence of ferrous metals (Aviram 1995). The lipid extracts of human atherosclerotic plaques have been shown to stimulate platelets. Once platelets are activated they change their shape, aggregate, and promote further coagulation. This leads to the formation of a clot called a thrombus that contains platelets, and fibrin. Depending on the location of the artery affected and the location of the blood vessel involved, it is the platelet and fibrin thrombus that suddenly obstructs blood flow precipitating angina, myocardial infarction and stroke.

The cycle is perpetuated as the activation of platelets causes the release of biochemical mediators that increases the attachment of oxidized LDL to blood vessel walls as well as the increased absorption of ox-LDL into macrophages, accelerating the rate of formation of foam cells (Nassar). Thus activated platelets with increased aggregation play a significant role in the development of atherosclerotic plaques as well as the formation of the thrombus that is associated with acute vascular obstruction such as occurs with coronary artery obstruction with subsequent infarction of the heart muscle called myocardial infarction.

Antioxidants such as glutathione can protect individuals on both an acute and chronic basis. Studies have shown that there is a decreased amount of glutathione in the reduced state in individuals suffering acute myocardial infarction compared to healthy individuals. Glutathione can also help protect LDL from oxidation not only by binding directly to the lipoprotein, but also following the accumulation of LDL in cells of the arterial wall. One mechanism involves the preservation of oxidation sensitive enzyme systems involved with the removal of lipoprotein, leading to the slowing of the process of atherosclerosis. The macrophage oxidation state depends on the availability of reduced glutathione (Aviram, 1998; Aviram 1996). The present invention provides a novel method for attenuation of the atherosclerotic process by providing reduced glutathione both to the systemic circulation of the individual and to the macrophage which needs adequate reducing capability in the form of reduced glutathione to enable the macrophage to remove oxidized LDL. The liposome of the current invention is readily absorbable into the general system and has preferential uptake by macrophages, which allows a further increase in the efficacy of the invention, and creating a novel method of preventing or slowing of atherosclerosis.

CRP has been demonstrated to be associated with increased platelet activation or stickiness and other components of increased coagulation and clotting (Fiedel). Because of the association of abnormal platelet aggregation with elevation of the CRP, the presence of an elevated CRP becomes an indication for the application of the invention.

Transition metals have been shown to be present in atherosclerotic plaques (Stadler). A transition metal is any of the thirty chemical elements 21 through 30, 39 through 48, and 71 through 80. This name comes from their position in the periodic table of elements, which represent the successive addition of electrons to the d atomic orbitals of the atoms as one progresses through each of the three periods. Transition elements are chemically defined as elements that form at least one ion with a partially filled subshell of d electrons. The transition elements have low ionization energies. They exhibit a wide range of oxidation states or positively charged forms. Thus, transition metals have the capacity to create excess free radicals from oxygen due to the fact that after losing electrons and becoming oxidized, they have an available orbital site for electrons from other elements such as oxygen. They net result is that transition elements can facilitate the formation of free radicals from oxygen, which are also known as reactive oxygen species.

The transition metals include iron, nickel, cadmium, zinc, copper, silver and mercury. Iron is involved in the early stages of atherosclerosis and the deposition of iron in the lining of arteries is closely associated with the progression of atherosclerosis (Ong). Published data from 11 countries indicate that the mortality from cardiovascular diseases is correlated with liver iron (Yuan). Clinical studies demonstrate that the combination of elevated LDL and transferrin saturation, a measure of iron in the body, is associated with an increased risk of coronary vascular mortality (Wells). Another transition metal, mercury, is also associated with an increased risk of heart and vascular disease including stroke (Salonen 1995, 2000).

It has been reported that the lining of the carotid artery in individuals with atherosclerotic lesions have been found to contain statistically significant increases in the amount of iron, particularly in the Fe(III) state as well as copper compared to healthy samples. Cholesterol levels were found to correlate with the amount of iron accumulation. The major products of lipid peroxidation, lipid hydroperoxides, undergo fission in the presence of transition metals, giving rise to the formation of peroxyl and alcoxyl radicals as well as aldehydes that are toxic to cells (Ong). The presence of these toxic materials leads to the further degeneration of the atherosclerotic plaque and facilitate the rupture of the plaque. Incubating cells with reduced glutathione has been shown to reduce the amount of lipid peroxide, and the addition of an iron chelator also diminishes the production of lipid peroxides in cell cultures (Thomas). The EDTA used in the invention has a high affinity for Fe(III), see Table 1, facilitating the removal of the harmful metal.

Alzheimer's disease may also be influenced by the combination of excess cholesterol and the accumulation of iron. In individuals 40 to 55 years of age increases in cholesterol levels from 181 to 200 almost tripled the odds for developing the amyloid protein deposits associated with Alzheimer's disease. One mechanism may be the increased accumulation of iron in the cells that line arteries that accompanies increases in cholesterol. In addition, recent evidence shows that metal ions such as zinc, copper, and iron are part of the mechanism of formation and toxicity of beta amyloid. It is advantageous in the mechanism of development of Alzheimer's disease to reduce both the cholesterol accumulation and the accumulation of excess iron and other transition metals.

The oxidation of LDL also compounds the vascular disease seen in diabetes. One of the mechanisms associated with oxidized LDL is interference with insulin signaling in macrophages (Liang). Insulin resistance, commonly seen in overweight individuals with the metabolic syndrome, is an important cause of both diabetes and increased atherosclerosis risk. Recent studies suggest that insulin resistance in the macrophage may be increased by the presence of oxidized LDL.

Oxidation stress has been shown to be a cause of increased insulin resistance in animals and deficiency of reduced glutathione has been shown to impair insulin induced glucose uptake, the hallmark of diabetes (Ogihara). Diabetic individuals have been shown to have hyper activation of platelets even when the diabetes is well controlled and there are no evident complications of their diabetes, apparently due to an underlying increase in oxidation stress (Vericel).

Parkinson's disease appears to have a similar mechanism of oxidation stress associated with an increased accumulation of transition metal such as iron. It appears that the substancia nigral cells may be particularly vulnerable to oxidation stress. Oxidation stress occurs in the substancia nigra cells because the metabolism of dopamine requires oxidation and can lead to the formation of free radicals from hydrogen peroxide formation. In the presence of metal ions such as iron the hydrogen peroxide can form hydroxyl ions, which can be very damaging to cells. The hydrogen peroxide is normally detoxified by reduced glutathione (GSH) in the reaction catalyzed by Glutathione peroxidase, thus an increased rate of dopamine turnover or a deficiency of GSH could lead to oxidative stress. Thus, it appears that free radicals, may be one of the important agents responsible for destruction of SN neurons, leading to PD.

Several studies have demonstrated a deficiency of the antioxidant biochemical reduced glutathione (GSH) in substantia nigra cells of individuals with Parkinson's disease. The magnitude of the reduction in GSH seems to parallel the severity of the disease.

The preferred embodiment of the invention that is EDTA and reduced glutathione encapsulated in a liposome creates a novel treatment of Parkinson's disease.

The use of the term “glutathione” or “glutathione (reduced)” will refer to glutathione in the reduced state.

A liposome is a microscopic fluid filled pouch whose walls are made of one or more layers of phospholipid materials identical to the phospholipid that makes up cell membranes. The object of this invention is the formation of liposomes made primarily of phosphatidyl choline, to be used orally for increasing the absorption of wag and reduced glutathione, allowing a systemic effect. Liposomes comprised of essential phospholipids have been used to carry drugs and similar materials into the general circulation of the body. The invention is the use of liposomes to carry reduced glutathione into the system.

Oral liposomal glutathione, reduced, in concentration of 2500 mg. per ounce was obtained from Biozone® Laboratories, Inc 580 Garcia Ave Pittsburg, Calif. 94565, USA.

In the invention uni and multilammellar liposomes created from lecithin are dispersed in a hydrocolloidal gel comprised primarily of glycerin. The glutathione in the reduced state are contained inside the liposome.

The liposome size ranges from 100 to 500 nanometer size.

Significant benefits are available to patient populations through the use of therapy with ethylene diamine tetraacetic acid (“EDTA”) in combination with glutathione encapsulated in a liposome to facilitate absorption, which have not been formerly appreciated. The combination and method can accelerate the evolution of improvement in a patient population that is affected by atherosclerosis.

Prior art discusses the efficacy of MgEDTA and Na₂EDTA in conjunction with atherosclerosis. However, other literature references numerous patient disorders associated with vascular problems, but has no reference to EDTA in combination with glutathione encapsulated in a liposome. There are a number of important areas in which this invention will have benefits:

Amelioration of atherosclerosis/vascular disease

Reduction of incidence of complication after vascular intervention such as angioplasty or other vascular surgery, including the slowing of restenosis

Reduction of incidence of stroke through anti-thrombin-platelet effect.

Therapy for patients with neurogenerative disorders associated with decreased vascular supply such as Alzheimer's disease

Transient ischemic attacks, usually from decreased blood flow to the brain

Memory loss and inability to concentrate.

Another benefit, not necessarily confined to those with disorders traditionally associated with aging, is to permit improvement of the condition of erectile dysfunction secondary to vascular insufficiency.

A different area of benefit is in the reduction of undesirable clotting resulting from sepsis or chronic infection.

Management of vascular complications of diabetes as well as facilitating the function of diabetic therapies such as insulin injections and oral agents for glycemic control.

### CASE EXAMPLES

Case 1

DH, a 60 year old man with elevated cholesterol and increased calcification in the coronary arteries on Ultrafast Computerized Tomogram radiologic study (Ultrafast CT), consistent with atherosclerosis.

Lab study showed cholesterol at 249 and platelet aggregation to be abnormal with a score of 5 (reference normal <4).

After 3 weeks using the LipoGSH and liposomal EDTA the patient noted that he felt better in general

Cholesterol score 209

Platelet aggregation 2-3 (reference range <4).

Case 2

DA, 35 year old man with fatigue and difficulty concentrating.

Platelet aggregation score was found to be 5 (reference normal <4).

After 2 weeks on a combination of liposomal glutathione 1 teaspoon daily (500 mg glutathione) and liposomal EDTA 500 mg twice a week the individual's platelet aggregation score became normal at level 3 (reference normal <4).

Case 3

Effect on glucose. metabolism: 56 year old woman with a 10 year history of diabetes requiring insulin therapy. At the time of using present invention she required

AM dose: 20 units of long acting insulin, 5 units regular insulin and glucophage (metformin) 500 mg tid

PM dose 15 units long acting insulin and 5 units regular insulin

This combination left the individual with stable levels of glucose with a range of 150 to 200 mg. upon arising in the morning.

On three occasions, 4 hours after ingesting 400 mg of the liposomal glutathione preparation alone, the patient developed a sudden drop in blood sugar, measuring levels of 50 mg glucose in the blood.

The listed series of ailments to which these benefits relate will be collectively referred to as vascular insufficiency, including the ailments which are secondary to vascular insufficiency such as erectile dysfunction, and including vascular insufficiency which is an effect of some other underlying etiology as in the case of sepsis.

While a recombinantly created analog of a naturally occurring human protein called protein C which has been given the trade name of Zovant, manufactured by Eli Lilly & Co., referenced in the “Wall Street Journal,” Jan. 4, 2001 issue page B2, could reduce undesirable clotting, the suggested price per dose is $5,000 to $10,000. Treatments with the present invention are several orders of magnitude cheaper per course.

Prior art, particularly, Rubin Martin, U.S. Pat. No. 5,114,974, May 19, 1992, suggests that EDTA can be efficacious in the amelioration of atherosclerosis. The proposed treatment in Rubin is to administer 3 grams of EDTA complex in a solution of 500 ml of 5% glucose (D₅W) for three hours. After administration, there would be three days rest. While the art suggests that zinc excretion in the urine will increase, no provision is made in the prior art to balance the associated body biochemistry with the increased zinc excretion as well as other essential trace elements, including Selenium, Manganese, Copper, Chromium and macronutrients to include Magnesium. The biochemical mechanism has been poorly understood and therefore no guidance is given as to how long treatment regimen should be continued nor are other components of biochemical imbalance addressed such as by this invention. For instance, the use of Na₂EDTA or MgEDTA causes depletion of zinc resources in the body, but the use of Zn+EDTA is not practical because the EDTA binding coefficent of Zn is higher than that of Na or Mg. See Table 1 showing the relative binding coefficients and illustrating the preferability of Na and Mg. The table is apparently adapted from Schwartzenbach, 1957. Scientific basis of EDTA chelation therapy, by Bruce Halsted, MD. Golden Quill Publishers, Inc. Box 1278, Colton, Calif. 92324.

TABLE 1

| | Stability | |
|---|---|---|
| | Constant | |
| Metal Cation | Log K | |
| Fe⁺⁺⁺ | 25.1 | Most Stable |
| Hg⁺⁺ | 21.8 | |
| Cu⁺⁺ | 18.8 | |
| Pb⁺⁺ | 18.5 | |
| Ni⁺⁺ | 18.0 | |
| Zn⁺⁺ | 16.5 | |
| Cd⁺⁺ | 16.5 | |
| Co⁺⁺ | 16.3 | |
| Al⁺⁺ | 16.1 | |
| Fe⁺⁺ | 14.3 | |
| Mn⁺⁺ | 13.7 | |
| Ca⁺⁺ | 10.7 | |
| Mg⁺⁺ | 8.7 | Least Stable |

Prior art by Kindness et al in US Patent application 20020182585 filed Dec. 5, 2002 references the use of EDTA in combination with glutathione given by intravenous infusion in the treatment of atherosclerosis and abnormal platelet aggregation. The prior art suggests that oral EDTA may be useful, but points out that the oral absorption of plain EDTA is low. The absorption of plain EDTA without the benefit of the present invention results in such a low absorption that there is no documented therapeutic effect. The administration of an oral, plain form of EDTA also does not gain the advantage referenced in the present patent of presenting the EDTA to macrophages by absorption of the liposome. There is also no reference in the prior art to the use of a liposome encapsulation to enhance the absorption of EDTA or glutathione to increase the therapeutic effect of the combination. There is also in Kindness Patent application 20020182585 no reference to the use of liposome encapsulation to deliver the glutathione systemically as referenced in the present patent.

Wagle, U.S. Pat. No. 6,770,663 refers to a method of treating atherosclerosis using thiazole, oxazole and imidazole compounds in combination with a statin or antioxidant such as glutathione and references the addition of EDTA as a preservative in the combination, there is no reference for the use of EDTA in therapeutic amounts in combination with glutathione in therapeutically active amounts encapsulated in a liposome for the treatment of atherosclerosis.

Smith in U.S. Pat. No. 6,764,693 references the use of liposomes designed to carry a combination of antioxidants including glutathione for the treatment of oxidation stress injury. However, there is no reference to the combination of EDTA and reduced glutathione. In Smith U.S. Pat. No. 6,764,693 there is also no reference for the treatment of vascular disease due to atherosclerosis nor a reference to the treatment of diabetes.

Demopoulos in U.S. Pat. No. 6,350,467 references the use of a pharmaceutically stabilized form of glutathione in a powdered form for the amelioration of effects of diabetes and vascular disease. There is no reference to the use of a liposome to deliver the glutathione and no reference to the enhanced absorption of glutathione into macrophage. There is no reference to the combination of glutathione and EDTA, which has advantages not considered in the Demopoulis U.S. Pat. No. 6,350,467.

Present therapy after vascular intervention procedures often involves the use of “blood thinners” like coumadin and aspirin, none of which are healthful as a long-term proposition, having various side effects which effects are often more pronounced in the very population most in need of the proposed therapy in this invention. The “blood thinners” have a significant side effect of permitting “blood leakage” in the brain, often leading to strokes. Non-steroidal anti-inflammatory drugs (NSAID's), including aspirin, do not have an effect on thrombin induced clotting of blood platelets.

The invention has the following general objects. Addressing these general objects will yield the benefits in addressing the disorders just mentioned. Those general objects are:

Minimization of inflammatory response after vascular incident or vascular intervention procedures

Improvement of immune system competency

Maintenance of proper body biochemistry, physiological function, including eliminating redox imbalance, and restoration of mineral balancing.

By increasing available glutathione, prevention of depletion of glutathione which depletion increases the propensity of “foam cells” to form and participate in arterial plaque formation.

By delivering the glutathione in a liposome, which has a preference for accumulation in liver and macrophages, two cell sites that are associated with the formation of atherosclerosis.

Reduction of plasma calcium with respect to plaque formation

The key anti-oxidant contemplated, which has a variety of positive biological effects is glutathione. The avoidance of a glutathione deficiency steers the patient to have a higher Th-1 response to Th-2 response ration that the patient would have with any glutathione deficiency. Peterson, J. et al, “Glutathione levels in antigen-presenting cells modulate Th1 versus Th2 response patterns,” Vol 95(6), Proceedings Nat'l Acad. Sci. USA p. 3071-76 (Mar. 17, 1998).

The background chemistry relates first to overall control mechanisms that rely on the regulation of “Free Radical Mechanisms” and the production of reactive oxygen species (ROS) and reactive oxygen intermediaries. Sources of ROS include heavy metals, pesticides, drugs, diet; activated leukocytes, enzymes, xenobiotics from indoor and outdoor air, for example—cigarette smoke, radon, O3, NO₂, SO2, car exhaust, x-rays, and ultraviolet, to name a few.

The shift of the oxidant/antioxidant balance, through free radical generation, in favor of oxidants in cells Is termed oxidative stress.

The term antioxidant is frequently used in medical literature, and should be correctly defined as “Any substance that, when present in low concentration compared to that of an oxidizable substrate, significantly delays or inhibits oxidation of that substrate.

This definition is particularly relevant to the use of EDTA and in particular its therapeutic window in association with supplemental antioxidant/anti-platelet therapy. Thus antioxidants can act at different levels in an oxidation sequence to prevent, intercept or to repair (reverse) cell and tissue free radical injury.

Examples of Intracellular Antioxidants are as follows:

Superoxide Dismutases

Catalase

Glutathione Peroxidase

Glutathione

Examples of extracellular antioxidants are as follows:

Vitamin E & Selenium

EDTA

Vitamin C

Uric Acid

Cholesterol

Albumin

Sulphydryls

β-Carotene

Molecular oxygen is a bi-radical, possessing two unpaired electrons in its outer orbital. In actively respiring cells, more than 90% of molecular oxygen is completely reduced by mitochondrial cytochrome oxidase in a four electron (Tetravalent pathway) with water as the end product.

The univalent and sequential reduction of O2 (univalent pathway), results in the formation of oxygen-derived free radicals.

The first reduction of O2 results in the formation of the superoxide anion radical. The subsequent reduction product is hydrogen peroxide (H2O2) which is a reactive oxygen intermediate (ROI) but does not have the structure of a radical.

Another reduction product of O2 is the hydroxyl radical which may result from the interaction of O2− with H2O₂ in the presence of iron (Fenton reaction). The hydroxyl radical is highly toxic and reacts immediately with most biological systems.

The last reduction of O2 results in the formation of water. To summarize:

O2∓e→O2.⁻Superoxide anion

O2 HO→HO.⁻+OH— Hydroperoxyl radical

HO+e∓H+→H2O2 Hydrogen peroxide

H2O2+e−→!OH+OH− Hydroxyl radical

Reactive oxygen species (ROS) are important mediators of cell and tissue injury (see figs.), and are the major players in the process of aging and apoptosis.

Thus oxygen-derived free radicals—superoxide anion, (O2.⁻), hydroxyl radicals OH.⁻ or metabolites such as hydrogen peroxide and hypochlorous acid (HOCl) must be regulated.

Activation of neutrophils is a natural part of the body defense mechanism. The activation generates O2.⁻ which is rapidly converted to H2O2by superoxide dismutase (SOD). However, it must be remembered that OH.⁻ formed non-enzymatically in the presence of Fe2+, or superoxide anion reacts with iron and copper to form hydroxyl radicals, Note: This is an important role for EDTA and other chelators as regulators of Fe/Cu generated free radicals. Also in neutrophils, myeloperoxidase results in the formation of HOCl from H2O2 in the presence of chloride ions. Additional protection is afforded by the combination of glutathione with EDTA in a liposome. One of the primary methods of removal of peroxide is the enzyme glutathione peroxidase. The peroxidase enzyme functions in conjunction with glutathione to transform the peroxide into harmless water.

Another free radical is produced during normal physiological processes, this is nitric oxide (NO). Nitric oxide is produced by the vascular endothelium and RELAXED vascular smooth muscle. It is also produced by phagocytes and epithelial lung cells whereby

the reaction may protect lung cells against (O2). However, O2.⁻+NO→ONOO.⁻−(PEROXYNITRITE), which is a strong oxidant and contributes to lung damage.

Excess (ONOO.⁻) may be produced when cytokines have increased production of both (NO) and (O2.⁻). At physiological pH peroxynitrate causes direct damage to proteins, and decomposes into toxic products that include nitrogen dioxide and hydroxyl radicals.

The multiple effects of nitric oxide particularly with respect to lung function emphasizes the role of free radicals in homeostasis, inflammation and oxidative stress.

In summary:

| | Oxygen species |
|---|---|
| Primary Reaction | |
| Superoxide Anion | (02.⁻) |
| Hydroxyl Radicals | (OH.⁻) |
| Hydrogen Peroxide | (H2O2) |
| Singlet Oxygen | (O.⁻) |
| Nitric Oxide | (NO) |
| Peroxynitrite | (ONOO.⁻) |
| Secondary Reactive | |
| Peroxyl Radical | (ROO.⁻) |
| Alkoxyl Radical | (RO.⁻) |

The benefits of anti-oxidant reactions can be summarized in the table below:

Antioxidant Actions

1. Prevent the formation of free radicals or initiation of peroxidation by scavenging free radicals.

2. Conversion of oxidant to less toxic free radicals.

3. Compartmentalization of reactive oxygen species away from vital cellular structures.

4. Repair of molecular injury induced by free radicals.

5. Binding of metal ions in forms that will not generate reactive species.

6. Removal of peroxides by conversion into non radical products such as alcohol.

7. Breaking chain reactions, i.e. reacting with chain propagating radicals (peroxyl and alkoxyl).

Background of Platelet Aggregation Assay

Antithrombotic and anticoagulant agents have been observed to be beneficial in the prevention of acute coronary events. While aspirin is the most widely used antithrombotic agent, aspirin interferes with only one of the pathways of platelet aggregation (thromboxane A2).

The aggregating stimuli unaffected or least modified by aspirin and/or associated NSAID:

1. ADP (ADENOSINE-5′-DIPHOSPHATE) and Collagen dependent aggregation pathway

2. Thrombin dependent pathway

3. Coagulation cascade

Current anticoagulation agents interfere only partially with the coagulation system and do not affect platelet aggregation. It is no surprise, therefore, that aspirin, plavix and anticoagulants cannot completely prevent coronary thrombotic events due to their limited mechanism of action.

High-risk patients are currently being advised to consider combination therapy with a Platelet inhibitor (aspirin or ticlopidine) and an anticoagulation agent such as heparin or coumadin. Only short-term therapy (1-3 weeks) with combination anticoagulants is recommended at this time.

Intravenous materials are under study to block thrombin related platelet aggregation, but so far no especially efficacious combination has been disclosed.

Important to the understanding of thrombin related platelet aggregation is the reaction of the blood platelet. The reaction of blood platelets is the single most important event in vascular disease. When the platelet undergoes insult or injury this causes a release reaction to take place resulting in the platelet undergoing both a change of shape and becoming sticky. In areas of reduced blood flow these cell fragments come together to form aggregates. The small aggregates migrate to larger vessels whereby they interact with plaque to form a blockage. Any inflammation aggravates the propensity to blockage. Several agents which are noticeably involved in this process can be readily monitored and the effects of EDTA on this process as well as the series of events involved in CAD/Atherosclerosis documented. Thus, by examining the following agents the series of events leading to cardiovascular events can be assessed:

Adenosine 5′ Diphosphate (ADP):

This substance is contained within storage granules within the platelet. Under stress, or in the presence of appropriate stimuli, ADP can be released from the platelet. This triggers the platelet to undergo the process of viscous metamorphosis (shape change-sticky) that results in more platelets adhering to each other. This process is reversible and is inhibited by EDTA as well as aspirin and other non-steroidal anti-inflammatory drugs (NSAID's). Thus, if an individual is self medicating either through the use of NSAID or Plavix by prescription, then ADP-induced platelet aggregation provides a monitor of therapy. Similarly the influence of various medications and their possible interaction with EDTA can effectively be overseen.

Epinephrine:

Epinephrine, like ADP, is contained within the storage granules. Under condition of stress, epinephrine release with the concomitant release of ADP will trigger the activation of clotting. Additionally, epinephrine from within the storage granules will contribute to the vasoconstrictive effects of catecholamines. As with ADP, the monitoring of epinephrine allows for the evaluation of aspirin NSAID effects. Vitamin B-complex and EDTA have a noted “calming” effect on these platelet responses.

Collagen:

The response of the blood platelet to the vessel wall is dependent on collagen. The collagen response is independent of calcium. However, if there is a blockage of the collagen receptor, the collagen-platelet effect will be inhibited. This phenomenon is also observed when the cell membrane becomes damaged as would occur in free radical pathology or with lipid peroxidation. The collagen response, independent of calcium, reflects membrane receptor integrity. Similarly, a good collagen platelet aggregation response means that in the event of trauma the platelets can form a hemeostatic plug. The collagen-platelet response also reflects the antioxidant efficacy of ascorbic acid, because Vitamin C is required for effective collagen cross-linking.

Thrombin:

The most important anti-thrombotic and anti-platelet effect of EDTA is seen in the inhibition of thrombin induced platelet aggregation. This test as such attests to the efficacy of EDTA in the treatment of vascular disease. How this is done will be addressed momentarily.

### PREFERRED MODE OF INVENTION

The preferred mode of the invention proposes the use of one of Na2EDTA, MgEDTA or calcium disodium EDTA (CaNa2-EDTA) in combination with reduced glutathione combined in a liposome. The preferred mode is preferably used in combination with the separate administration of intermittent oral zinc and selenium therapy. The surprising, though logical, effect that is yielded by the combination is that the reduction in inflammatory response, and/or increase in immune system competency, increases the effectiveness of the combination and enables better patient recovery. This is further enhanced by the decreased likelihood of glutathione depleted foam cells. That recovery can be objectively ascertained by measurement of the glutathione level and by performing a platelet aggregation test. Those tests can be performed immediately prior to treatment. If performed after administration of a dose, then benefit will be seen, but the best means of measuring efficacy of the treatment is to measure glutathione level and platelet aggregation immediately before commencement of administration of the next dose. If improvement is noted at that time, and prior to commencement of subsequent doses, then a positive trend can be ascertained. The suggested dose for an average adult is 500 mg of EDTA combined with GSH 1200 mg in 0.5 ounce of the liposome combination delivered 3 times per week. On the next day after the EDTA/glutathione liposome administration, a zinc supplement of 25 mg-50 mg should be administered orally.

Dosing in patients with decreased kidney function is calculated using the Cockcroft-Gault Equation,

$$Creatinine Clearance ( CrCl ) = \frac{( 140 - Age ) \times Wt in Kg . )}{( 72 \times serum Creatinine )}$$

EDTA dose administered at each ingestion=50 mg EDTA per (Weight in Kg)×(CrCl/100), up to a maximum of 3.0 g EDTA. The volume of the invention sufficient to deliver the quantity of EDTA is then administered with the glutathione dose contained in the amount calculated for EDTA.

Abbreviations:

CrCl=computed renal glomerular filtration rate in ml/min

Age=patient's age

Cr=serum creatinine in mg/dL

For women, multiply the above result by 0.85

Oral administration of EDTA has been generally over looked as the absorption of EDTA in a “neat” fashion that is without the use of the described invention for increasing absorption is low. It is suggested that the invention be administered one hour after a meal to prevent EDTA from binding essential minerals from foods and minimizes the likelihood of nutrient deficiency. The term pharmaceutically acceptable carrier includes oral, aerosol, intravenous, topical (transdermal) and transmucosal both oral and rectal and other acceptable routes of administration through which the liposome containing the combination of EDTA and glutathione may be administered.

The addition of glutathione yields another effect in this invention not facially evident from the independent properties of the basic components of the invention. The glutathione cycle is a critical body cycle whose importance has not been fully appreciated. Administration of glutathione in a liposome allows the insertion of glutathione into critical cells and organs the liposome has a preferential uptake into liver, lung, spleen, and macrophage. Increasing the availability of glutathione particularly in the macrophage and also the spleen, enhances the immune system competency of the patient and reduces the inflammation associated with diseases like atherosclerosis and infection.

Additionally, selenium should be administered orally on the next day after treatment in the amount of 200 μg to maintain adequate levels. Selenium is an important catalyst for glutathione peroxidase activity in the glutathione cycle enabling the capture and excretion of free radicals, especially hydroxyl radicals.

For a procedure such as angioplasty or other invasive vascular surgery, the reduction of inflammation and inhibition of thrombotic effect and platelet aggregation effect will accelerate the evolution of improvement in the patient's condition, and defer the onset of symptoms as well as in disorders not requiring or unable to be treated by invasive vascular procedures. In the instance of sepsis, the reduction of inflammation and inhibition of thrombotic effect and platelet aggregation effect will accelerate the evolution of improvement in the patient's condition, and defer the onset of destabilizing symptoms.

In the best mode, there should be measurement of lipid peroxide level and/or glutathione level and a platelet aggregation test, as well as prothrombin time, activated partial thromboplastin time, total serum calcium, ionized calcium, total magnesium, ionized magnesium, β2-microglobulin and serum creatinine or creatinine clearance. The glutathione level, is very difficult at most laboratories. The measurement of platelet aggregation, is not available at most laboratories. All other tests are routinely available in clinical laboratories. Measurement of glutathione can be done through a difficult process according to Tietze.

### METHOD OF MONITORING THE USE OF THE INVENTION IN THE TREATMENT OF INCREASED PLATELET AGGLUTINATION AND VASCULAR DISEASE

The invention proposes semi-weekly to weekly monitoring of glutathione and platelet aggregation as treatment commences for two weeks, and then, assuming a non-negative trend in glutathione level and platelet aggregation, bi-weekly monitoring for three months, and thereafter bi-weekly or monthly monitoring. Creatinine should be monitored initially and then every tenth administration of EDTA. The inventors also propose pre-treatment with the liposomal combination of the invention prior to any invasive vascular intervention procedure.

The purpose of the invention is to ultimately restore platelet aggregation characteristics to normal levels as set forth in the attached Table I entitled Coagulation Profile. A user should be mindful that creatinine clearance should be monitored to confirm proper kidney function. Because each patient can have unique characteristics and profiles, a baseline is suggested of a complete blood count, the preferred indicia of which are in the attached table II. A baseline chemistry profile including a Comprehensive Metabolic Profile, is also suggested per the attached Table III, and a baseline Lipid/Cardiac Risk is also suggested per the attached Table IV. Table V has a baseline chart to use for determining efficacy of kidney function for purposes of the invention. Table VI has a post-treatment chart of preferred biochemical results. Abbreviations should be known to those skilled in the art. Table VII is added in order to assist with abbreviations.

The inventors also recommend the monitoring of HDL cholesterol and LDL cholesterol and ferritin. See, Table VIIIA and VIII B. Mitigation of abnormal levels closer to normal ranges enables further evaluation of a patient's progress, though not as precisely predictive as the platelet aggregation indication tests set out in this invention.

Glutathione Level Test:

Determination of glutathione levels for plasma and/or red blood cells is the preferred test. The test is performed according to Tietze, 1968 Enzymic Method for the Quantitative Determination of Nanogram Amounts of Total and Oxidized Glutathione Analytical Biochemistry with an additional reference of Tietze, 2^{nd} ed., Chemical Chemistry 1994, pp. 1779-1780. This Tietze method has been modified as follows:

GR
GSSG+DTNB+H⁺66 2G−SH+DTN⁺ (1)
GS⁻+ (2)

where GSSG is glutathione, oxidized

GR is glutathione reductase

DTNB is a sulfhydryl reagent 5,5′-dithiobis-(2-nitrobenzoic acid)

G−SH is glutathione, reduced

DTN⁺ is dithiobisnitrobenzoic acid

GS⁻ is a transition state between glutathione reduced and oxidized

The method of glutathione assay provides a sensitive method for total and oxidized glutathione. The modification increases sensitivity for spectrophotometric analysis. The reagents in use throughout this invention, including for this test, are either generally available from a chemical supply house or available from Sigma Chemical Co., Inc. or a company associated with it, Aldrich Chemical Company, of St. Louis, Mo. Incorporating DTNB, a sulfhydryl reagent 5,5′-dithiobis-(2-nitrobenzoic acid) in the first reaction which possesses a molar absorption at 412 mμ then forms two moles of GSH per mole of reduced nucleotide utilized in the GSSG reduction in reaction (2). The rate of chromophore development depends on the concentration of glutathione in the reaction mixture detectable to 10 nanograms ml⁻¹. This provides a highly sensitive and specific procedure for measuring glutathione. The normal level should be approximately 200-400 micromoles/liter for plasma and red blood cells. The test may be performed on an automated clinical chemistry analyzer (also called a random access analyzer) such as Roche Cobas Fara. Samples are collected carefully to prevent contamination. Frozen plasma collected from ACD, EDTA, and heparin may be used. The invention could test reduced glutathione but there is not any efficacy over testing total glutathione. Another means of testing glutathione is specifically referenced in Ellerby, L. et al, Measurement of Cellular Oxidation, Reactive Oxygen Species, and Antioxidant Enzymes During Apoptosis, 322 Methods in Enzymology 419-420 (Academic Press 2000).

A discussion of the therapeutic value of appropriate levels in the glutathione pathway is discussed in Rahman I, MacNee W, Free Radical Biological Medicine 2000, May 1, 28(9): 1405-1420.

Testing for thrombic propensity and propensity to platelet aggregation:

A blood sample is taken and stabilized to prevent natural clotting. The blood is centrifuged. It is spun relatively slowly at approximately 1000 rpm for 15 minutes (360 g (g=gravitational constant) so red blood cells (“RBC”) are at bottom leaving platelets suspended in plasma just above RBC's. The platelets are then pipetted off trying to have no RBC's in the pipette. The platelet rich plasma is then ready for testing. The pipetted platelets are tested with five different reagents in a cuvette comparable in size to a major artery. The reagents are:

1) ADP

2) Epinephrine

3) Collagen

4) Thrombin

5) the patient's blood with saline as a control

The cuvettes containing the platelets and selected reagent are run in a platelet aggregometer supplied by Helena Laboratories of Beaumont Texas referred to as a Kyoto Daiichi Kogaku Co. Ltd. (“KDK”) model Monitor IV Aggregation Recorder for 5 minutes. A magnet is placed in each tube before starting the timer to rotate the mixture in the cuvette and provide turbulence to imitate normal turbulence of blood flow.

After five minutes a printout is generated reflecting the five cuvettes. The graph runs from 0-5 minutes. The purpose of the graph is to show the relative aggregation during the five minute run. The reading is from 0-100% based on the clarity of the solution with 0 being the most turbid due to free platelets. Light in the aggregometer is less scattered if platelet aggregation occurs. The purpose of the visual inspection is to insure that if a large thrombus is blocking light, the test result is competent. A further purpose is to support a clinical opinion.

The laboratory makes a visual observation of the separated platelet sample run with the reagent thrombin, selecting from the following list the best description, and when appropriate microscopic examination of the test cuvette contents.

1) Fibrin strand

2) Small single thrombus

3) Large single thrombus

4) Small fibrin clot

5) Large fibrin clot

6) Free floating single thrombus, with free platelets observed

7) Free floating fibrin strand, with free platelets observed

8) Single balloon thrombus with no free platelets

9) Free platelets-no clotting

Scoring system: 1-5 with 1 being the best response to respective challenge and 5 representing poor response to respective challenge.

The most commonly observed states are the following three: free platelets—no clotting, single thrombus, or large fibrin clot.

The printout is examined and interpreted to produce a score of one to five for the platelet sample, with one being the best score for patient health, and five the worst. One is the best rating and normally evidenced by positive graph results and free platelets-no clotting. The graphs, and a review of the primary diagnosis for the patient's sample, and known patient medication or graphical observations, enable the score to be adjusted for drug interactions such as a patient taking aspirin which may influence test results.

The graph produces a line that begins at a low point at the commencement of the time and may then be straight or normally curved upward, or some combination of the two.

A general guide for reading the results is as follows: If the test is done on a pre-treatment sample, and the graph line for thrombin is straight, and there is a fibrin clot or a large thrombus, then the patient is rated a five.

The most desirable result, and one showing successful treatment, is a straight line for the ADP, epinephrine and thrombin reagents, and observed free platelets. Such a result is rated a one. This implies inhibition of aggregation.

If the line is straight with respect to collagen, then the sample is usually rated a 4 due to interference with the collagen receptor. If there is a fibrin clot or a large thrombus, and a straight line for the thrombin reagent, then the patient usually receives a score of 4 or 5. If the graph for thrombin shows a biphasic curve, then the result is average and usually graded a three. In that instance, the observation of the cuvette is usually that fine to medium aggregates are seen in the samples run with reagents ADP, epinephrine or collagen. In the cuvette having the sample run with thrombin, a single thrombus is normally seen in that instance. In certain instances, there is a straight line in the sample run with thrombin that then rises and forms a curve. That shows a delay >60 sec. in inhibition of aggregation with a biphasic response and usually rates a score of 2-3. This is often seen in a post-treatment sample-after a treatment but prior to completion of therapy.

In general, if the thrombin graph line is not straight and there is no visual observation with clot-free platelets, there is usually a fibrin clot or a single large thrombus suggesting a rating of four to five and the patient should continue the therapy described in this invention.

For a separated platelet sample in a cuvette with the collagen reagent, if there is a straight line, and other data or the tube observations suggest a propensity to aggregation, those results suggest that a drug interaction such as acetaminophen, e.g. Tylenol (registered trademark of Johnson & Johnson) or aspirin may be present. If the line for ADP rises and then proceeds in a straight line, this indicates the patient is under oxidative stress, and the patient is scored as a 4 unless other data shows a more negative score is appropriate.

If a rise of curve is relatively smaller for and then straight for the ADP or epinephrine reagents, the sample usually is scored a 3. A yet smaller rise could be scored as a 2.

If there is any visual observation aside from free platelets, or a single large thrombus, and, for instance the graph as to the thrombin reagent proceeds 20-40-60-80 this implies a delay in the onset of thrombin induced aggregation.

Ideally, lines that show no thrombus or fibrin clot in the thrombin reagent cuvette, and a lack of aggregation, meaning a reading of zero, and where there is visual observation of free platelets, the patient should have a favorable rating of one. Straight lines that show only free platelets in ADP and epinephrine also rate a most favorable patient health rating and show inhibition of epinephrine-induced or ADP-induced platelet aggregation.

The control will show that the mechanical components of the machine are working properly because there should be no alteration in platelet function by the addition of a small amount of saline. No alteration in platelet aggregation should be apparent in that cuvette.

Saline additionally acts as a patient control whereby if the test platelets are subject to spontaneous aggregation, e.g. “stress type” reactions, this will be evidenced by the formation of aggregated platelet clumps. This response being abnormal reflects an increased propensity for clot formation.

This description and the directions for the KDK Monitor IV Aggregation Recorder, which are incorporated by reference, should enable a reasonably skilled person to determine the success of treatment and the success during the course of treatment by measuring platelet aggregation.

The term “therapeutic dose” is intended to mean that amount of a drug or pharmaceutical agent that will elicit the biological or medical response of a tissue, a system, animal or human that is being sought by a researcher, veterinarian, medical doctor or other clinician. The term “prophylactically effective amount” is intended to mean that amount of a pharmaceutical drug that will prevent or reduce the risk of occurrence of the biological or medical event that is sought to be prevented in a tissue, a system, animal or human by a researcher, veterinarian, medical doctor or other clinician. A consideration of these factors is well within the purview of the ordinarily skilled clinician for the purpose of determining the therapeutically effective or prophylactically effective amount.

The concept and invention is not meant to be limited to the disclosures, including best mode of invention herein, and contemplates all equivalents and permutations to the invention and similar embodiments to the invention for humans and mammals and veterinary science. Equivalents include all pharmacologically active racemic mixtures, diastereomers and enantiomers of the listed compounds and their pharmacologically acceptable salts.

TABLE I
BASELINE-COAGULATION PROFILE

| TEST | NORMAL RANGE | UNITS |
|---|---|---|
| Prothrombin Time PT | 11-15 | sec. |
| Activated Partial Thromboplastin Time | 16-25 | sec. |
| (APPT) | | |
| Fibrinogen | 200-400 | m/dL |

Platelet Aggregation Score:

| | | Normal score |
|---|---|---|
| | | under invention |
| Reagent | | criteria |
| Adenosine 5′ | ADP | 3 or less |
| diphosphate | | |
| Epinephrine | EPI | 3 or less |
| Collagen | Coll | 3 or less |
| Thrombin | THR | 3 or less |

TABLE II
BASELINE-CBC/Complete Blood Count

| TEST | NORMAL RANGE | UNITS |
|---|---|---|
| WBC | 5-10 | Thou/CMM |

| | | | |
|---|---|---|---|
| RBC | M: 4.6-6.2 | F: 4.2-5.4 | M/μL |
| HgB | M: 14-18 | F: 12-16 | g/dL |
| HCT | M: 40-54 | F: 37-47 | % |

| | | |
|---|---|---|
| MCV | 82-99 | FL |
| MCHC | 33-36 | g/dL |
| RDW | 11.5-14.5 | |
| PLT | 150-400 | k/μL |
| MPV | 6.2-10.8 | FL |
| Lymph | 25-40 | % |
| Mono | 1-8 | % |
| Baso | 0.5-1.0 | % |
| Eosin | 1-4 | % |
| Segs | 50-70 | % |

TABLE III
BASELINE-SMAC/Metabolic Comprehensive Profile

| TEST | NORMAL RANGE | UNITS |
|---|---|---|
| Alkaline Phosphatase | 30-103 | U/L |
| Bun | 7-19 | mg/dL |
| Creatinine | 0.7-1.4 | mg/dL |
| Bun/Creatinine | | |
| Glucose | 64-112 | mg/dL |
| Total Protein | 6.0-8.2 | g/dL |
| Uric Acid | 2.1-6.1 | mg/dL |
| Albumin | 3.8-5.2 | g/dL |
| Albumin/Globulin Ratio | | |
| Calcium | 8.5-10.1 | mg/dL |
| Phosphorus | 2.4-4.2 | mg/dL |
| Sodium | 136-145 | mmol/L |
| Potassium | 3.5-5.1 | mmol/L |
| Chloride | 95-106 | mmol/L |
| Bilirubin | 0.0-1.0 | mg/dL |
| LDH | 100-220 | U/L |
| SGOT | 12-29 | U/L |
| SGPT | 9-41 | U/L |
| GGT | 11-55 | U/L |

TABLE IV
Baseline-Lipid/Cardiac Risk

| TEST | NORMAL RANGE | | UNITS |
|---|---|---|---|
| Cholesterol | 140-200 | | mg/dL |
| Triglycerides | 40-160 | | mg/dL |
| HDL - Cholesterol | see chart | | mg/dL |
| LDL - Cholesterol (Calc) | see chart | | mg/dL |
| Ferritin | see chart | | ng/dL |
| Apolipoprotein A1 | M: 115-190 | F: 115-220 | mg/dL |
| (APO A1) | | | |
| Apolipoprotein B | M: 70-160 | F: 60-150 | mg/dL |
| (APO B) | | | |

| | | |
|---|---|---|
| APO B/APO A1 Ratio | <1.0 | |
| Lipoprotein-a | 15-30 | mg/dL |
| Homocysteine | 4.0-15.0 | μmole/L |
| Fibrinogen | 200-400 | mg/dL |
| CPK | 41-186 | u/L |

TABLE V
BASELINE-Renal Profile
Creatinine Clearance

Specimen Date:
Patient:

| Patient | Measurement | Units |
|---|---|---|
| Patient Height | | Inches |
| Patient Weight | | Pounds |
| Patient Surface Area | | Square meters |
| Specimen Collection Time | | Hours |
| Urine Volume | | Millilters |
| Plasma Creatinine | | mg/dL |
| Urine Creatine | | mg/dL |

Corrected Creatinine Clearance mL/min

| TEST | NORMAL RANGE | UNITS |
|---|---|---|
| β2-Microglobulin | 0.85-1.62 | mg/L |

TABLE VI
Post Tx (Treatment) 1

| | NORMAL RANGE | |
|---|---|---|
| TEST | (Invention score) | UNITS |
| Platelet Aggregation | (Score 3 or less) | |
| ADP | (Score 3 or less) | |
| EPI | (Score 3 or less) | |
| COLL | (Score 3 or less) | |
| THROMBIN | (Score 3 or less) | |
| PT | 11-15 | sec. |
| APTT | 16-25 | sec. |
| Magnesium | 1.2-2.3 | mEq/L |
| Calcium | 8.5-10.1 | mg/dL |
| Magnesium (ION) | 1.5-2.3 | mg/dL |
| Calcium (ION) | 3.9-5.5 | mg/dL |

Index of Abbreviations:

WBC=white blood cell

RBC=red blood cell

HgB=hemoglobin B

HCT=hematocrit

MCV=mean corpuscular volume

MCHC=mean corpuscular hemoglobin concentration

RDW=red cell distribution width

PLT—platelets

MPV=mean platelet volume

Lymph=lymphocytes

Mono=monocytes

Baso=basophylls

Eosin=eosinophylls

Segs=segmented neutrophylls

FL=femtoliter

SGOT(ALT)=Serum glutamate oxaloacetate transaminase Alanine Aminotransferase

SGOT (AST)=Serum glutamate oxaloacetate transaminase Aspartate Aminotransferase

BUN=blood urea nitrogen

LDH=lactate dehydrogenase

GGT=gamma glutamyltransferase

CPK=Creatine Kinase

M=10⁶ per ml³

K=10³ per ml³

TABLE VIIIA

| HDL CHOLESTEROL (mg/dL) | | | LDL CHOLESTEROL (mg/dL) | | |
|---|---|---|---|---|---|
| AGE (YRS) | MALE | FEMALE | AGE (YRS) | MALES | FEMALES |
| 0-14 | 30-65 | 30-65 | 0-19 | 60-140 | 60-150 |
| 15-19 | 30-65 | 30-70 | 20-29 | 60-175 | 60-160 |
| 20-29 | 30-70 | 30-75 | 30-39 | 80-190 | 70-170 |
| 30-39 | 30-70 | 30-80 | 40-49 | 90-205 | 80-190 |
| over 40 | 30-70 | 30-85 | 50-59 | 90-205 | 90-220 |
| | | | 60-69 | 90-215 | 100-235 |
| | | | over 70 | 90-190 | 95-215 |

Values for African-Americans about 10 mg/dL higher

TABLE VIIIB
FERRITIN

| | | | | |
|---|---|---|---|---|
| MALES | 18-45 years | 22-340 ng/dL | >45 years | 22-415 ng/dL |
| FEMALES | 18-45 years | 6-115 ng/dL | >45 years | 15-200 ng/dL |

### REFERENCES

Dr John Banas, The Dorothy & Lloyd Huck Chair, Chairman of the Department of Cardiovascular Medicine at Morristown Memorial Hospital in Morristown, N.J., and Professor of Clinical Medicine, College of Physicians & Surgeons, Columbia University, New York, N.Y., at September 2002, symposium on vascular disease,

Aviram M. Macrophage foam cell formation during early atherogenesis is determined by the balance between pro-oxidants and anti-oxidants in arterial cells and blood lipoproteins. Antioxidants & redox signaling. 1999 Winter;1(4):585-94 PMID: 11233155

Aviram M, Fuhrman B. LDL oxidation by arterial wall macrophages depends on the oxidative status in the lipoprotein and in the cells: role of prooxidants vs. antioxidants. Molecular and cellular biochemistry. November 1998;188(1-2):149-59 PMID: 9823020

Aviram M. Interaction of oxidized low density lipoprotein with macrophages in atherosclerosis, and the antiatherogenicity of antioxidants. European journal of clinical chemistry and clinical biochemistry : journal of the Forum of European Clinical Chemistry Societies. August 1996;34(8):599-608 PMID: 8877334

Aviram M. LDL-platelet interaction under oxidative stress induces macrophage foam cell formation Thrombosis and haemostasis. July 1995;74(1):560-4 PMID: 8578524

Kaplan M, Aviram M. Oxidized low density lipoprotein: atherogenic and proinflammatory characteristics during macrophage foam cell formation. An inhibitory role for nutritional antioxidants and serum paraoxonaseClinical chemistry and laboratory medicine August 1999;37(8):777-87 PMID: 10536926

Nassar T, Sachais B S, Akkawi S, Kowalska M A, Bdeir K, Leitersdorf E, Hiss E, Ziporen L, Aviram M, Cines D, Poncz M, Higazi A A. Platelet factor 4 enhances the binding of oxidized low-density lipoprotein to vascular wall cells. The Journal of biological chemistry. Feb. 21, 2003;278(8):6187-93. Epub Dec. 03, 2002. PMID: 12466273

Ogihara T, Asano T, Katagiri H. Sakoda H, Anai M, Shojima N, Ono H, Fujishiro M, Kushiyama A, Fukushima Y, Kikuchi M, Noguchi N, Aburatani H, Gotoh Y, Komuro I, Fujita T. Oxidative stress induces insulin resistance by activating the nuclear factor-kappa B pathway and disrupting normal subcellular distribution of phosphatidylinositol 3-kinase. Diabetologia. May 2004;47(5):794-805. Epub May 01, 2004 PMID: 15127200

Ong, W, Halliwell B. Iron, atherosclerosis, and neurodegeneration: a key role for cholesterol in promoting iron-dependent oxidative damage?Annals of the New York Academy of Sciences. March 2004;1012:51-64. PMID: 15105255

Rother E, Brandl R, Baker D L, Goyal P, Gebhard H, Tigyi G, Siess W.

Subtype-selective antagonists of lysophosphatidic Acid receptors inhibit platelet activation triggered by the lipid core of atherosclerotic plaques. Circulation. Aug. 12, 2003;108(6):741-7. E. PMID: 12885756

Salonen J T, Seppanen K, Nyyssonen K, Korpela H, Kauhanen J, Kantola M, Tuomilehto J, Esterbauer H, Tatzber F, Salonen R

Intake of mercury from fish, lipid peroxidation, and the risk of myocardial infarction and coronary, cardiovascular, and any death in eastern Finnish men. Circulation. Feb. 1, 1995;91(3):645-55. PMID: 7828289

Salonen J T, Seppanen K, Lakka T A, Salonen R, Kaplan G A. Mercury accumulation and accelerated progression of carotid atherosclerosis: a population-based prospective 4-year follow-up study in men in eastern Finland. Atherosclerosis. February 2000;148(2):265-73. PMID: 10657561

Stadler N, Lindner R A, Davies M J. Direct detection and quantification of transition metal ions in human atherosclerotic plaques: evidence for the presence of elevated levels of iron and copper. Arteriosclerosis, thrombosis, and vascular biology. May 2004;24(5):949-54. Epub Mar. 04, 2004 PMID: 15001454

Thomas J P, Geiger P G, Girotti A W. Lethal damage to endothelial cells by oxidized low density lipoprotein: role of selenoperoxidases in cytoprotection against lipid hydroperoxide- and iron-mediated reactions Journal of lipid research. March 1993;34(3):479-90 PMID: 8468531

Vericel E, Januel C, Carreras M, Moulin P, Lagarde M. Diabetic patients without vascular complications display enhanced basal platelet activation and decreased antioxidant status. Diabetes. April 2004;53(4):1046-51. PMID: 15047620

Wells B J, Mainous A G 3rd, King D E, Gill J M, Carek P J, Geesey M E. The combined effect of transferrin saturation and low density lipoprotein on mortality. Family medicine. May 2004;36(5):324-9. PMID: 15129378

Yuan X M, Li W. The iron hypothesis of atherosclerosis and its clinical impact. Annals of medicine. 2003;35(8):578-91. PMID: 14708968

What is claimed is:

1. A combination for amelioration of vascular insufficiency, comprising: In a liposome encapsulation a therapeutic dose of reduced glutathione and EDTA.

2. The method according to claim 1, further comprising:
A therapeutic dose of zinc.

3. The method according to claim 1, further comprising:
A therapeutic dose of Selenium.

4. A method of treatment of vascular insufficiency, comprising:
Combining in a liposome encapsulation therapeutic doses of reduced glutathione and EDTA; and
Administering said doses in said encapsulation to a mammalian patient.

5. The method according to claim 4, further comprising the following step: Administering a therapeutic dose of Zinc.

6. The method according to claim 5, further comprising the following step: Administering a therapeutic dose of Selenium.

7. A method of treatment of a mammalian patient at risk for increased platelet aggregation, or at risk for or experiencing increased coagulation, comprising:
Combining in a liposome encapsulation therapeutic doses of reduced glutathione and EDTA; and
Administering said doses in said encapsulation to a mammalian patient.

8. The method according to claim 7, further comprising the following step: Administering a therapeutic dose of Zinc.

9. The method according to claim 8, further comprising the following step: Administering a therapeutic dose of Selenium.

10. The method according to claim 4, further comprising: the following step:
Administering a therapeutic amount of liposome encapsulation of reduced glutathione and EDTA to an individual documented by testing to have increased platelet aggregation; and
Repeatedly administering therapeutic amounts of said reduced glutathione and EDTA to achieve and maintain platelet aggregation in the normal range.

11. The method according to claim 10, further comprising the following step: Administering a therapeutic dose of zinc.

12. The method according to claim 11, further comprising the following step: Administering a therapeutic dose of selenium.

13. Administration of a therapeutic amount of liposome encapsulation of reduced glutathione and EDTA to an individual at risk for or experiencing increased coagulation.

14. The method according to claim 13, further comprising the following step: Repeated administration of the therapeutic amounts to achieve and maintain platelet aggregation in the normal range.

15. The method according to claim 14, further comprising the following step: Administering a therapeutic dose of zinc.

16. The method according to claim 15, further comprising the following step: Administering a therapeutic dose of selenium.

17. Administration of a therapeutic amount of liposome encapsulation of reduced glutathione to an individual with low glutathione.

18. The method according to claim 17, further comprising the following: continued administration to achieve and maintain normal glutathione levels.

19. The method according to claim 5, further comprising the following step: Administering a therapeutic dose of Selenium.

20. Administration of a therapeutic amount of liposome encapsulation of reduced glutathione and EDTA to an individual with elevation of transition metal such as lead.

21. The method according to claim 20, further comprising the following step: Administering a therapeutic dose of Zinc.

22. The method according to claim 21, further comprising the following step: Administering a therapeutic dose of Selenium.

23. The method according to claim 20, further comprising the following step: Repeated administration of the therapeutic amounts to achieve and maintain reduction in the toxic metal level to normal.

24. The method according to claim 23, further comprising the following step: Administering a therapeutic dose of zinc.

25. The method according to claim 24, further comprising the following step: Administering a therapeutic dose of selenium.

26. Administration of a therapeutic amount of liposome encapsulation of reduced glutathione and EDTA to an individual with Parkinson's disease.

27. The method according to claim 26, further comprising the following step: Administering a therapeutic dose of Zinc.

28. The method according to claim 27, further comprising the following step: Administering a therapeutic dose of Selenium.

29. The method according to claim 26, further comprising the following step: Repeated administration of the therapeutic amounts to achieve and maintain reduction in symptoms of Parkinson's disease toward normal.

30. The method according to claim 29, further comprising the following step: Administering a therapeutic dose of zinc.

31. The method according to claim 30, further comprising the following step: Administering a therapeutic dose of selenium.

32. Administration of a therapeutic amount of liposome encapsulation of reduced glutathione to facilitate glucose moderating therapies in an individual with diabetes.
